(19) United States
(12) Patent Application Publication
(10) Pub. No.: US 2005/0238587 A1
(43) Pub. Date: Oct. 27, 2005

(54) Method for suppressing mold

(76) Inventors: Donald P. Dennis, Atlanta, GA (US); Walter Charles Hayhurst JR., Centennial, CO (US)

Correspondence Address: LAW OFFICE OF SANFORD J ASMAN, 570 VININGTON CT, DUNWOODY, GA, 30350, US

(21) Appl. No.: 10/977,424

(22) Filed: Oct. 29, 2004

Related U.S. Application Data
(62) Division of application No. 10/660,832, filed on Sep. 12, 2003.
(60) Provisional application No. 60/417,314, filed on Oct. 9, 2002.

(51) Int. Cl. A61K 35/78

(52) U.S. Cl. 424/40; 424/736; 424/745; 424/750; 424/769

(57) ABSTRACT

The method for suppressing mold consists of extract from a citrus seed, citrus skin, or other botanical mold suppressant, with the preferable suppressant being made of grapefruit seed extract combined with propylene glycol. The mold suppressant has been formulated to provide for air borne administration, preferably by using a fogging machine, such as a glycol fogger, which may be of the type generally used to apply insecticides, or for special effects in which a “fog” machine is used by entertainers.

### CROSS-REFERENCE TO RELATED APPLICATIONS AND PATENTS

The present application is based upon provisional patent application Ser. No. 60/417,314 filed Oct. 9, 2002.

### BACKGROUND OF THE INVENTION

The present invention relates to a method and formulation for suppressing mold.

The growth of mold (fungi) in structures, including homes, has become an officially-recognized source of serious illness. Mold, e.g., black mold (stachybotrys chartarum), has been found to cause flu-like and allergy-like symptoms. Skin rashes, inflammation of the respiratory tract, bloody noses, fever, headaches, neurological problems, and suppression of the immune system have all been linked to exposure to black mold. Its presence causes serious health problems, especially among children, and it has been blamed for several deaths. Various factors, including excessive moisture due to structural and drainage problems, encourage mold growth. In residential and commercial structures mold has been found to grow within walls, in carpeting and underlying padding, on sheetrock, and in insulation in walls and ceilings. Certain synthetic “stucco” construction materials have been found to trap moisture behind walls, resulting in a perfect breeding ground for mold. There have been numerous lawsuits involving mold growth in structures as a result of water leaks and improper drainage.

The Atlanta Journal-Constitution ran a two part article entitled “Sick Buildings/A Special Report” on Jul. 20-21, 2003, in which illnesses, including rashes, sinus problems, flu-like conditions, and chronic respiratory illness were among the health problems associated with indoor mold. The article cited a 1994 study in which Dr. Dorr Dearborn and Dr. Ruth Etzel, an epidemiologist with Atlanta's Center for Disease Control and Prevention (“CDC”) noted a pattern of pulmonary hemorrhage in Cleveland infants which were attributed to stachybotrys chartarum and other molds. The article further cited a 1998 study by Dr. Eckardt Johanning, a physician at the Mount Sinai School of Medicine in which he studied 151 patients who had been exposed to mold and found about half of them had central nervous system complaints, such as concentration problems, dizziness, and fatigue.

While all researchers are not convinced that there has been adequate study of the relationship of mold to illness, 17 states have introduced legislation related to mold, and there is a bill pending in Congress to study health problems caused by mold, and the CDC has asked the Institute of Medicine, a national advisory group, to study the medical damage from mold.

While some people do appear to be bothered by mold more than others, all varieties have the potential to cause illness. Mold triggers allergic reactions, asthma attacks, fungal infections in the lungs of people with chronic medical conditions, and hypersensitivity pneumonitis, an inflammation of the lungs. Some molds, e.g., Stachybotrys chartarum, are known to produce toxins that can be inhaled. In 1999 doctors at the Mayo Clinic concluded that mold may be responsible for a majority of the sinus infections in the United States.

As mold has been found to affect people in a variety of detrimental ways, a simple, effective, inexpensive method for suppressing mold growth and/or eliminating mold would be very desirable.

### SUMMARY OF THE INVENTION

In accordance with the present invention, a formulation and method for administration of the formulation have been developed. In particular, the formulation uses a substance which has been determined to be an active mold suppressant. In the preferred embodiment of the invention, grapefruit seed extract is used as the active mold suppressant. However, other substances, including the extract of the seeds of other citrus fruits (e.g., lemon, lime, tangerine, orange, etc.), the extract of the skin of citrus fruits (e.g., grapefruit, lemon, lime, tangerine, orange, etc.), and certain other substances, including oregano oil, oregano extract, lavender extract, olive leaf extract, and tea-tree oil may be used.

In accordance with the preferred method of the present invention, the formulation is preferably administered by a fogging process. In particular, the formulation is preferably administered by placing a fogging machine in the room, or other area, to be treated and administering a fog of the mold suppressant over a period of time. While the use of a (so-called “glycol”) fogger is preferred, other dispersal means which are capable of dispersing an aerosol or fog of the mold suppressant of the present invention in a substantially air borne application can be used, as well. As used herein the term “air borne application” is intended to mean that the mold suppressant remains suspended in the air for an extended period of time, rather than merely being passing through the air for direct application to a surface at which a spray is directed. The intent is that particles of the mold suppressant be of such small size and of such great density that they are able to fully permeate all surfaces and cavities within the “area” (actually the volume) being treated. Sprays, on the other hand, merely treat the specific areas to which the sprays are applied. To further augment the application, it has been found that a heated fog results in particles which are able to rise within wall cavities and other enclosed areas, e.g., HVAC ducts.

Particular advantages of the “glycol fogging” process of the present invention are that the formulation includes propylene glycol which has a dehydrating effect on the mold, in that it helps dry out the mold. Further, the application process involves heating the formulation, and the heated fog actually rises up within internal wall and HVAC cavities (ducts and returns).

### BRIEF DESCRIPTION OF THE DRAWING

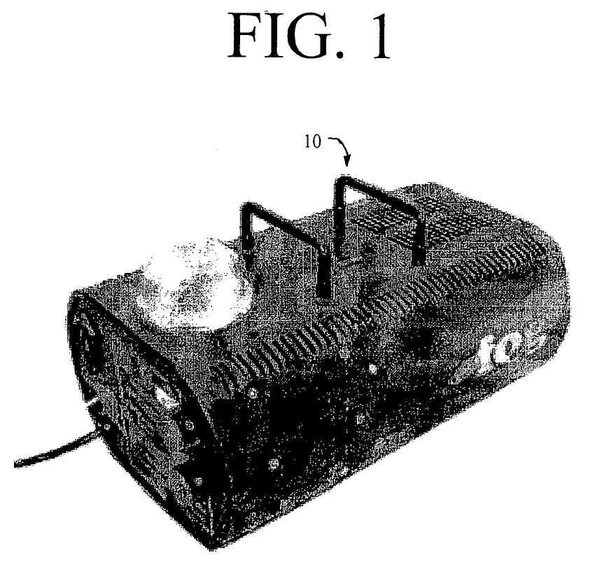

In the sole FIGURE of the Drawing FIG. 1 is a pictorial view of the fogger used in the preferred embodiment of the present method invention.

### DETAILED DESCRIPTION OF THE PREFERRED EMBODIMENT

In accordance with the present invention, a formulation has been developed which has the effect of suppressing and/or eliminating mold. The preferred embodiment of the invention has been specifically formulated to provide for flash evaporation of the substance in a fog containing small size particles which are capable of penetrating HVAC filters, duct work, household items, clothing, computer and electronic equipment, and other items typically found in homes and/or businesses. The formulation, when administered in an air borne “fog” is able to disperse within, and rise up to the top of wall cavities, and it covers large areas more quickly than other application means. In addition, the formulation of the preferred embodiment of the invention has a dehydrating effect on mold, and it has a long lasting effect on mold suppression, provided that any water intrusion, which has the effect of reintroducing both mold and a medium for mold growth, is stopped.

In the preferred embodiment of the invention, the formulation uses grapefruit seed extract as an active ingredient in a formulation which has been designed to be administered in an air borne “fog”. While grapefruit seed extract is used in the preferred formulation, other substances which have also been found to act to suppress or kill mold may be used in lieu of the grapefruit seed extract. These other substances include, but are not limited to the extract of the seeds of other citrus fruits (e.g., lemon, lime, tangerine, orange, etc.), the extract of the skin of citrus fruits (e.g., grapefruit, lemon, lime, tangerine, orange, etc.), and certain other substances, including oregano oil, oregano extract, lavender extract, olive leaf extract, and tea-tree oil, any of which may be used without departing from the scope of the present invention.

One particular formulation which has been showed to be particularly effective in the present application is made up in 4000 ml (4.0 liter) quantities as follows:
30 cc of a mixture composed of 60% natural grapefruit quaternary compound and 40% Vegetable Glycerin USP
770 cc distilled water
3200 cc Propylene Glycol (also known as 1,2-Propanediol; 1,2-Dihydroxypropane; Methylethylene glycol; Trimethyl glycol; 1,2-Propylene glycol; monopropylene glycol; propane-1,2-diol; alpha-propyleneglycol; dowfrost; PG 12; sirlene; solar winter ban; or Propancdiol) USP.

The foregoing formula will yield a total formulation amount of 4000 ml (4.0 liters). As will be recognized by those skilled in the art, while distilled water is substantially without impurities, it is not necessary to actually use distilled water. Accordingly, one should understand that while distilled water is preferred, it may be replaced by water having no impurities which inhibit the efficacy of the present invention.

In accordance with the preferred embodiment of the present invention, the 30 cc mixture set forth above contains the grapefruit seed extract in the form of a 60% natural grapefruit quaternary compound and 40% Vegetable Glycerin USP. The preferred version of that mixture is sold by Bio/Chem Research, 925 Lakeville Street No. 206, Petaluma, Calif. 94952 under the trademark “Citricidal”.

In accordance with the preferred embodiment of the method of the present invention, the area to be treated is subjected to a “fog” of the mold killing and suppressing formulation of the present invention. Referring to FIG. 1, the sole FIGURE of the drawing, a commercial fogger, such as the Lite F/X fogger 10, sold by Wal-Mart is used in the preferred embodiment of the inventive method to suppress and kill mold. While other commercially available foggers can be used, the Lite F/X fogger has been found to work quite well, while being quite simple to operate. As will be understood by those skilled in the art, a feature of fogging devices is that they use heat to create a “fog” having very small droplets which are readily air borne throughout the area being treated.

On the other hand, there are other foggers which have features absent in the Lite F/X, which may make them desirable for use in particular applications. For example, there are foggers which enable the operator to adjust the particle (droplet) size which may be useful for fogging particular sites. In particular, larger droplets help the fog settle faster, without as much drift, while smaller droplets are better for most indoor fogging applications in that they allow the smaller droplets to “float” (stay suspended in the air) longer in order to get into “hidden” areas. One such fogger, the Flex-A-Lite Model 2600 fogging machine made by B & G Equipment Company, P.O. Box 130, 6120 N. Rt. 611, Plumsteadville, Pa. 18949 (Phone 215-766-8811, Fax 215-766-8240), provides an adjustable flow rate of from about 0 to 9 ounces per minute, and it could be used to carry out the method of the present invention where adjustable flow rates and particle sizes would be desirable. Another fogger, the Flex-A-Lite Model 2600, includes a flexible hose which allows an operator to direct the fog (from the nozzle at the end of the flexible hose) into specific areas, as desired, and it also includes a flow adjustment knob which allows the operator to adjust the liquid flow rate to dispense materials at average droplet sizes under 20 microns (for a water based formulation at room temperature). Droplets of this size remain suspended in the air for extended periods of time, so they disperse into areas otherwise difficult to reach and treat. At higher flow rates, the average droplet size tends to increase to about 80 microns when the liquid flow is adjusted for maximum output. The larger sized droplets are dispersed by air flow and can settle on surfaces, which makes them particularly useful for coating the inside of heat ducts and similar items. As will be understood by those skilled in the art, the use of such other foggers would not depart from the spirit or scope of the present invention.

While the preferred embodiment of the present formulation has been disclosed, as has the preferred fogger, it will be understood by those skilled in the art that the formulation can be varied for different applications, so long as it uses a sufficient amount of mold suppressant formulation (preferably, grapefruit seed extract, as set forth above) to perform the desired function of mold suppression and/or elimination. While the preferred embodiment of the invention uses a commercially available grapefruit seed extract, in the form of Bio/Chem Research's “Citricidal Professional Strength GSE” product, other grapefruit seed extract mixtures may be used. By way of example, one could use a mixture comprised of:
80% Propylene Glycol (1,2-Propanediol)
18.5% Water
1.5% Grapefruit Seed Extract

Similarly, other formulations with greater, or lesser amounts of grapefruit seed extract can be used. Thus, suitable ranges for the mixture may contain:
40%-90% Propylene Glycol
1%-30% water
0.005%-59% Grapefruit Seed Extract

Alternatively, depending on the consistency, volatility, and viscosity of the particular grapefruit seed extract, up to 100% grapefruit seed extract may be used, or, as set forth above, other mold suppressants disclosed herein, can be used.

With respect to the method of application, the preferred method is to fog a room, or other area, using a so-called “glycol” fogger. In particular, the Lite F/X Fog Model 1741 glycol fogger, sold at Wal-Mart, is used in the preferred embodiment of the method for suppressing mold disclosed herein. However, any other fogging machine, e.g., the Lite F/X Commander Fog, the My-T-Lite Model 2300 fogger, or the Flex-A-Lite Model 2600 fogging machine, each made by B & G Equipment Company, could be used without departing from the present invention. The important aspect of the “fogger” lies in its ability to adequately disburse the formulation such that any mold present will be treated. Glycol foggers operate by injecting a water/glycol mixture into a preheated metal chamber (“heat exchanger”) which causes the fluid to flash-evaporate. The heated fog is carried throughout the area being treated, rising into cavities where conventional spraying is ineffective.

Thus, while it is preferable to use a glycol fogger, other devices, such as a humidifier, either a standalone one, or one connected to a central heating ventilating and air conditioning (“HVAC”) system, could be used, as could a room humidifier, vaporizer, nebulizer (e.g., ultrasonic nebulizers), or any similar device capable of providing an aerosol or mist of the grapefruit seed extract to treat the relevant area. However, as it is important to dry out mold in order to suppress and kill it, the synergistic combination of propylene glycol, which acts as a drying agent, and the heated fog which rises and permeates the area being treated, are considered to be quite important to the invention. Further, while water is used in the preferred formulation, as mold thrives in a moist environment, the amount of water should not exceed about 30% of the formulation. When more water is used, it has been found that mold suppression is limited, and if an excessive amount of water is used, mold growth is actually encouraged.

While excessive fogging is not harmful, as the grapefruit seed extract is rated generally recognized as safe (“GRAS”), propylene glycol which is present can be harmful, so those applying the present formulation by fogging should wear protective masks, such as standard carbon (activated charcoal) chemical masks, and protective goggles, during the fogging. Once the visible fog has dissipated, it is safe to enter the area which has been fogged, but to be especially cautious, it is recommended that one wait for between twenty-four and thirty-six hours after the fogging has been completed, as that time allows the fog, along with any residual materials and smells, to completely dissipate.

The fogging, itself, can be conducted using approximately one liter of formulation for every 4500 square feet of area being fogged. A typical fogging operation takes about ten minutes for a 600 square foot area.

The present inventive method of fogging is distinguished from the spraying of various dilutions of Citricidal or other grapefruit seed extract mixes in that the formulation disclosed herein uses a unique mix which allows for flash evaporation of the suppressant in a fog containing small size particles which are able to penetrate HVAC filters, duct work, household items, clothing, and computer and electronic equipment. Further, the present formulation, when applied using the present inventive method, in which a heated fog is produced, results in air borne particles which rise in wall cavities and are able to cover large volumes more quickly than other application means. Further, it is believed that the presence of propylene glycol have both a dehydrating and a cleansing effect on the mold, so that it results in a long lasting mold suppression effect, provided, of course, that any water intrusion is stopped.

In accordance with the present invention, various procedures have been found to work well in different applications. While these procedures are exemplary, they are not intended to limit the invention.

For fogging HVAC systems and wall cavities it has been found effective to fog at full output all intakes of the HVAC system until at least ⅓ of the room is affected by the fog. This usually takes from 20 to 30 minutes. Then, it is preferred to continue to fog individual rooms at approximately 80% capacity throughout the structure for an additional 20 minutes to 1 hour depending on the severity of the contamination and the room volume. The area is then vacated and kept closed up for 12 to 36 hours after fogging. After treatment, it is preferable to retest for mold. Then, if the mold test comes back positive, the baseboard of walls should be removed where the mold test was positive, and small holes should be drilled to allow for swab testing to determine if there is mold inside the wall cavities. If so, then holes should be drilled at the base of the wall between studs and small holes should be drilled at the top of the wall. Fog should be slowly administered into the wall cavity through these openings at 30 to 40% of capacity for 4 to 5 hours or until any adjoining rooms are impacted by the process. It is preferable to then have the treated walls and interior insulation removed and disposed of in an approved manner, then refog prior to, and following, construction of the new wall.

A preferred manner for treating vehicle HVAC and interior cabins involves adjusting the vehicle's fan to its high speed position, then turning the air intake control so that it pulls air from outside. Then turn the air conditioner on, and fog at full into the vehicle's air intake (which can be determined at the windshield vent using a tissue to see where it is sucked down). Fogging is then conducted at the full setting until the car is full of fog. This process is repeated using each intake. After the car is full of fog the air conditioning system is turned off, and then the fogging process is repeated for at least 20 minutes, or more if there was an excessive amount of contamination.

In order to treat clothing and household items, all clothing should be hung on hangers with a small space between each article of clothing. Mattresses and/or highly stuffed sofas which are badly contaminated, should be disposed of, as they cannot be successfully decontaminated with fogging. Lightly contaminated mattresses should be placed in a vertical position, then the fogger should be set to its full output setting and the room or closet should be fogged for 30 minutes to about an 1 hr. depending on contamination severity.

Aircraft are fogged using their air conditioning intake either from inside the aircraft or from the outside using the air supply docking hose as an input port. Depending on the severity of the contamination, aircraft can generally be fogged in from about 20 minutes to about an hour after the cabin has been filled with fog.

A series of tests were run in which a Lite F/X Fog machine, sold by Wal-Mart was the fogging machine which was used to disperse the mold suppressant of the preferred embodiment of the present invention in the tests whose description follows.

Before and after each treatment, tests were conducted to determine the level of mold present. When testing rooms, Petri dishes of Saboraud Dextrose agar (“SDA”) which was specifically formulated to grow mold from spores, while excluding bacterial growth, were used. The test plate or dish was opened in the test area for one hour, after which the lid was replaced and securely taped in place to prevent the entry of extraneous materials into the test dish.

When testing clothing, a “slap” test was used in which the Petri dishes containing the SDA agar were opened and then slapped against the fabric three times, with the expectation that spores would be jarred off the fabric and onto the agar.

Following exposure, each of the Petri dish samples was incubated for 5 days at 25 degrees Celsius. Duplicates were sent to two additional test labs to check for result consistency. Thus, each test was conducted using three samples, with each sample being analyzed by a separate laboratory. After 5 days, the samples were examined, and the total number of mold colonies growing on the agar were tabulated. The tests were repeated on a schedule of 1, 2, 7, 14, and 20 days.

In addition to the mold tests, which were intended to test the efficacy of the mold killing ability of the formulation of the preferred embodiment of the present invention, additional tests were run to determine whether any extraneous chemicals were present, or were added to the test environments. To accomplish these tests, a Dragger tube arrangement was used. A Dragger tube test is a vacuum test that pulls air through a tube into a cotton sampler. The sample, which is retained in the cotton, is analyzed for chemical content. This test was run in one hour increments for 12 hours, and then daily for 5 days. Chemicals in the air before the tests began were expected to still be there after treatment in accordance with the present invention. That expectation was borne out, in that chemicals, such as carbon monoxide, pesticide, and paint residues which were found prior to the testing also appeared in the same quantities after testing as they appeared prior to treatment with the mold suppressant, as described herein. The only exception to the foregoing was that in one test 0.1 ppm of dimethyl benzene was present after the application of mold suppressant, but it had completely dissipated or was not found to be present one hour after mold suppressant treatment.

### Test 1—Five Houses

The first test involved five houses which each had a prior history of high mold spore count. Prior to treatment, tests were performed to measure the pre-treatment mold spore counts using SDA mold spore gravity test plates. The pre-treatment mold spore counts were in the TNTC (“Too Numerous To Count”) range.

Treatment, using the formulation of the preferred embodiment was conducted. The fogger's timer was set to a setting of 6, and the fogger was left on for 20 minutes within the closed space of the test site. This applied a volume of 115 cc of the mold suppressant. No effort was made to direct the fog within the test site, i.e., the fog was simply allowed to disperse.

There were an average of five rooms per house. Following treatment in accordance with the present invention, no mold colonies were present in the SDA test plates which were exposed one day after treatment, two days after treatment, or seven days after treatment in any of the homes.

Two of the homes showed a single mold spore colony on the SDA test plates which were exposed fourteen days after treatment, while the other three homes continued to show no mold spore colonies.

Based upon the foregoing tests of houses treated in accordance with the present invention, it is believed that all mold spore colonies which were present prior to the testing were killed, but that mold spores were reintroduced into two of the houses post-treatment.

### Test 2—Office Building

The second test was performed in an office building which had a history of water intrusion. Prior to treatment, tests were performed to measure the pre-treatment mold spore counts using an SDA mold spore gravity test plate. The pre-treatment mold spore counts were in the TNTC (“Too Numerous To Count”) range.

The test was performed by treating six rooms of the building in the same manner as Test 1 above. Following treatment there were zero mold spore colonies, as measured by SDA plates one, two, seven, and fourteen days after treatment.

### Test 3—Clothing

The third test was performed on clothing. Prior to performing this test, each garment was subjected to exposure for thirty days in a known “moldy” environment. In particular, the garments were hung on hangers in the homes used in Test 1, above. Thereafter, they were treated after being arranged in the following ways:
(a) On hangers, uncovered;
(b) On hangers, covered in plastic wrap of the type used by dry cleaners;
(c) Folded, on a shelf;
(d) Folded, in open dresser drawers; and
(e) In closed cardboard boxes.

Prior to treatment, all of the clothing had mold spore colonies (using the SDA gravity plate tests) which numbered TNTC. Following treatment the results were as follows:
(a) Clothes treated while hanging, uncovered, were found to have zero mold spore colonies 1, 2, and 14 days following treatment, and 1 colony twenty days after treatment;
(b) Clothes treated while hanging, covered in plastic wrap of the type used by dry cleaners, were found to have 123 mold spore colonies 1 day after treatment; 124 mold spore colonies 2 days after treatment, and 100 mold spore colonies 14 and 20 days after treatment;
(c) Clothes treated while folded, on a shelf, were found to have zero mold spore colonies 1, 2, and 14 days after treatment, and 2 mold spore colonies 20 days after treatment;
(d) Clothes treated while folded, in open dresser drawers, were found to have 500 mold spore colonies 1 day after treatment; 490 mold spore colonies 2 days after treatment; 494 mold spore colonies 14 after treatment; and 499 mold spore colonies 20 days after treatment; and
(e) Clothes treated while in closed cardboard boxes continued to have mold spores too numerous to count (“TNTC”) 1, 2, 14, and 20 days after treatment.

The results of the clothing tests confirmed that treatment in accordance with the present invention is effective with respect to articles of clothing which were exposed to the mold suppressant, while less effective with respect to articles which were “protected” from exposure to the mold suppressant. The more the articles were separated from the mold suppressant, the less effective the treatment actually was, with the clothing in closed boxes apparently untreated, while the clothing in open drawers was only partially treated, and the clothing hanging in dry cleaner bags was somewhat more treated.

### Test 4—Vehicles

The fourth test was performed in vehicles, as it has been found that automotive environments are harmful to people who are mold sensitive, and it has been found that automotive ventilating systems can be especially prone to harboring mold colonies. In this test four cars and one truck were tested. Prior to testing, Automobile 1 was found to have had 124 mold spore colonies; Automobile 2 was found to have had 234 mold spore colonies; Automobiles 3 and 4 were each found to have had 500 mold spore colonies; and the single Truck was found to have had 23 mold spore colonies.

Each of the vehicles was found to have zero mold spore colonies 1, 2, and 14 days after treatment.

### Test 5—Ductwork

The fifth test was performed in ductwork used in heating/ventilating/air conditioning (“HVAC”) systems, as it has been found that air circulation in homes, and buildings which have ductwork causes mold to settle in the ductwork. Unless the ductwork is treated, it has been found that if the mold present in the ductwork is left untreated, it will re-contaminate the structure.

The ductwork which was tested was that in the five houses which were the subject of Test 1, above. Prior to treatment, the SDA tests showed mold spore colony counts of 311 (House 1), 205 (House 2), 199 (House 3), 155 (House 4), and 111 (House 5).

The mold spore colony counts were found to be zero 1, 2, and 14 days after treatment in each house.

As will be understood by those familiar with the art, it is very important to remove moisture from areas in which mold is being suppressed, as mold thrives in a moisture rich environment. Accordingly, one of the benefits of the preferred embodiment of the mold suppressant formulation disclosed herein is that it uses propylene glycol which has a dehydrating effect on any mold which is present. Those skilled in the art will recognize that other substances may be substituted for the propylene glycol, but propylene glycol also has the further benefit of being a material which works particularly well with the fogger apparatus used in the preferred method of application, as described herein.

1-8. (canceled)

9. The method of suppressing and killing mold comprising dispersing fine particles of a mold suppressant formulation in an area to be treated using an apparatus which provides for air borne dispersal of said mold suppressant formulation.

10. The method of suppressing and killing mold of claim 9 wherein said apparatus which provides for air borne dispersal of said mold suppressant formulation is one which generates an aerosol, a fog, a fine spray, or mist of air borne mold suppressant.

11. The method of suppressing and killing mold of claim 10 wherein said apparatus which provides for air borne dispersal of said mold suppressant formulation is selected from the group consisting of foggers, ultrasound foggers, and humidifiers.

12. The method of suppressing and killing mold of claim 9 wherein said apparatus which provides for air borne dispersal of said mold suppressant formulation is a fogger selected from the group consisting of commercially available foggers.

13. The method of suppressing and killing mold of claim 9 wherein said apparatus which provides for air borne dispersal of said mold suppressant formulation is a fogger selected from the group consisting of the Lite F/X Fog, the Lite F/X Commander Fog machine, the My-T-Lite Model 2300 fogger, and the Flex-A-Lite Model 2600 fogging machine.

14. The method of suppressing and killing mold of claim 13 wherein said apparatus which provides for air borne dispersal of said mold suppressant formulation is a Lite F/X fogger.

15. The method of suppressing and killing mold of claim 14 wherein said apparatus which provides for air borne dispersal of said mold suppressant formulation is a Lite F/X fogger, and said mold suppressant formulation comprises:
(a) 0.005% to 98% of 60% natural grapefruit quaternary compound and 40% vegetable glycerin;
(b) 1.0 to 20% distilled water; and
(c) 2% to 98% propylene glycol.

16. The method of suppressing and killing mold of claim 14 wherein said apparatus which provides for air borne dispersal of said mold suppressant formulation is a Lite F/X fogger, and said mold suppressant formulation comprises:
(a) 0.75% of 60% natural grapefruit quaternary compound and 40% vegetable glycerin;
(b) 19.25% distilled water; and
(c) 80% propylene glycol.

17. The method of suppressing and killing mold of claim 16 wherein said fogger is used to disperse approximately one liter of mold suppressant formulation for every 4500 square feet of area to be treated.

18. The method of suppressing and killing mold of claim 16 wherein said fogger is operated for about ten minutes for each 600 square feet of area to be treated.
